(19) United States
(12) Patent Application Publication
(10) Pub. No.: US 2026/0108219 A1
(43) Pub. Date: Apr. 23, 2026

(54) HEARTBEAT DETECTING HANDCUFF DEVICE

(71) Applicant: Christopher Vaughn, Las Vegas, NV (US)

(72) Inventor: Christopher Vaughn, Las Vegas, NV (US)

(21) Appl. No.: 18/923,248

(22) Filed: Oct. 22, 2024

(51) Int. Cl. A61B 5/00; A61B 5/024; E05B 75/00

(52) U.S. Cl. CPC A61B 5/746; A61B 5/02438; A61B 5/6802; A61B 5/7405; A61B 5/7455; E05B 75/00

(57) ABSTRACT

A heartbeat detecting handcuff device for monitoring the heartbeat of a detainee includes a pair of handcuffs which can be placed on a detainee's wrists by a law enforcement officer for restraining the detainee. A pair of heartbeat monitors is each integrated into a respective one of the pair of handcuffs. Each of the pair of heartbeat monitors can monitor the detainee's heartbeat when the pair of handcuffs are worn on the detainee's wrists. Each of the pair of heartbeat monitors includes an audible alert which emits an audible alarm when a respective heartbeat monitor detects a heartbeat that is displaying abnormalities. In this way the audible alert can audibly alert the law enforcement officer that the detainee is in need of medical attention.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to handcuff devices and more particularly pertains to a new handcuff device for monitoring the heartbeat of a detainee. The device includes a pair of handcuffs which can be worn on a detainee's wrists and a pair of heartbeat monitors which are each integrated into a respective handcuff for detecting the detainee's heartbeat. Each of the pair of heartrate monitors includes an audible alert which emits an audible alert when the heartbeat monitors detects an abnormality in the detainee's heartbeat. In this way a law enforcement officer is notified that the detainee is in need of medical attention.

### (2) Description of Related Art Including Information Disclosed under 37 CFR 1.97 and 1.98

The prior art relates to handcuff devices including: a handcuff device that includes an electrical shock element for delivering an electric shock to a detainee under pre-determined conditions; a handcuff device that includes handcuffs attached together by a resilient wire cable and a monitoring device integrated into the handcuffs for monitoring a detainees physiological state; and a handcuff device that includes a pair of handcuffs and a respiration unit integrated into the handcuffs for monitoring a detainee's respiration and a heartrate sensor for sensing the detainee's heartrate and a communication unit integrated into the handcuffs for broadcasting an output signal to a law enforcement officer. In no instance does the prior art disclose a handcuff device that includes a pair of handcuffs and a pair of heartrate monitors each integrated into a respective handcuff for monitoring the heartrate of a detainee and an audible alert which emits an audible alert when the pair of heartrate monitors senses an abnormality in the detainee's heartrate and light for emitting light and a vibration unit for producing a haptic response.

### BRIEF SUMMARY OF THE INVENTION

An embodiment of the disclosure meets the needs presented above by generally comprising a pair of handcuffs which can be placed on a detainee's wrists by a law enforcement officer for restraining the detainee. A pair of heartbeat monitors is each integrated into a respective one of the pair of handcuffs. Each of the pair of heartbeat monitors can monitor the detainee's heartbeat when the pair of handcuffs are worn on the detainee's wrists. Each of the pair of heartbeat monitors includes an audible alert which emits an audible alarm when a respective heartbeat monitor detects a heartbeat that is displaying abnormalities. In this way the audible alert can audibly alert the law enforcement officer that the detainee is in need of medical attention.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of a heartbeat detecting handcuff device according to an embodiment of the disclosure.

FIG. 2 is a front phantom view of a handcuff of an embodiment of the disclosure.

FIG. 3 is a right side view of a handcuff of an embodiment of the disclosure.

FIG. 4 is a back phantom view of a handcuff of an embodiment of the disclosure.

FIG. 5 is a perspective in-use view of an embodiment of the disclosure.

### DETAILED DESCRIPTION OF THE INVENTION

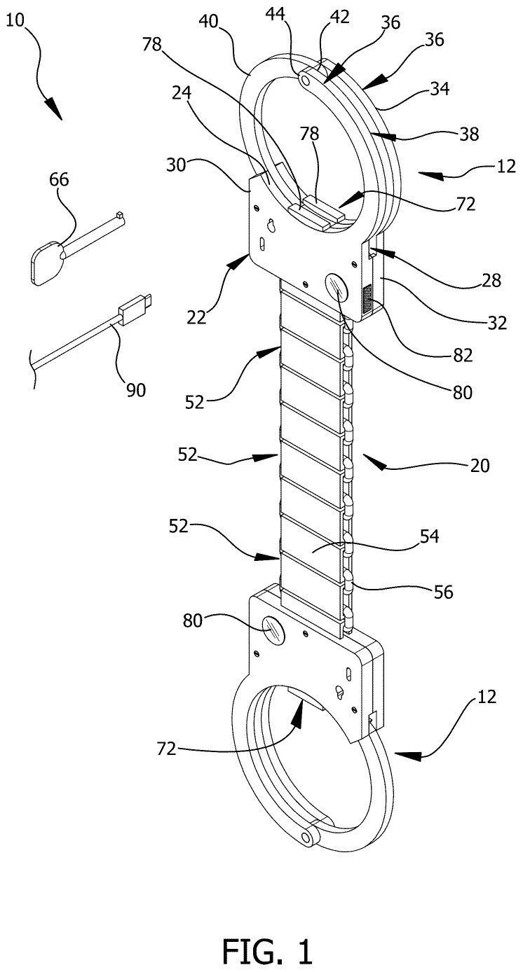

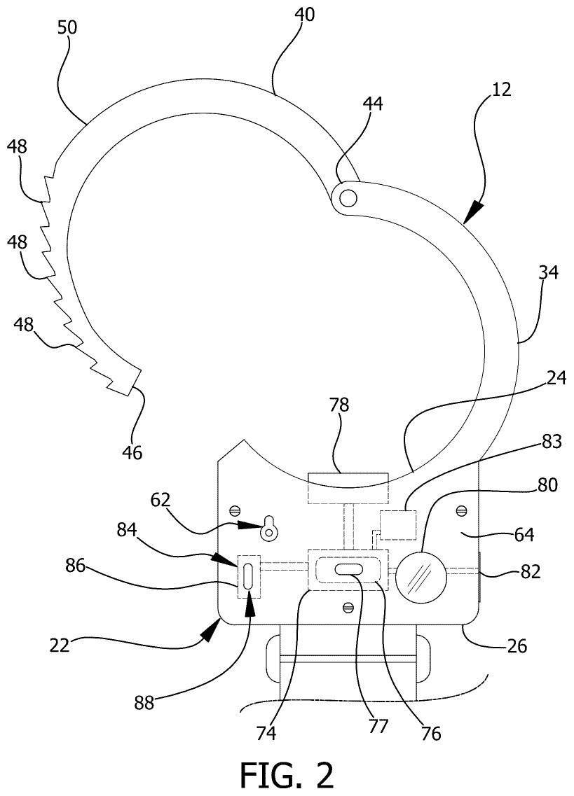

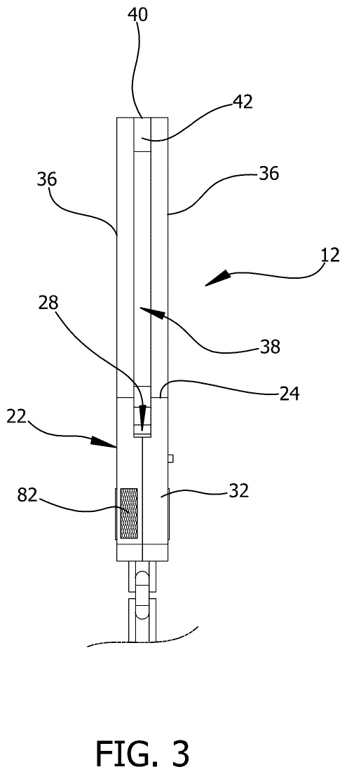

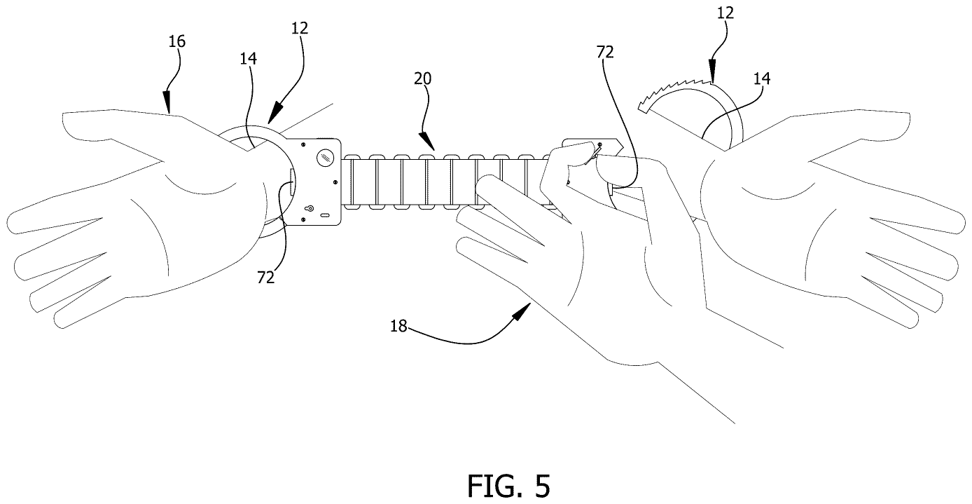

With reference now to the drawings, and in particular to FIGS. 1 through 5 thereof, a new handcuff device embodying the principles and concepts of an embodiment of the disclosure and generally designated by the reference numeral 10 will be described.

As best illustrated in FIGS. 1 through 5, the heartbeat detecting handcuff device 10 generally comprises a pair of handcuffs 12 which can each be placed on a respective wrist 14 of a detainee 16 by a law enforcement officer 18 for restraining the detainee 16. The pair of handcuffs 12 may comprise handcuffs of any conventional design that are approved for use by law enforcement officers, including but not being limited to municipal police officers, state highway patrol officers and federal law enforcement officers. The pair of handcuffs 12 includes a chain 20 which extends between the pair of handcuffs 12 to secure the detainee's wrists 14 together when the pair of handcuffs 12 is placed on the detainee's wrists 14. Additionally, the pair of handcuffs 12 may be attached together with a hinge or other similar type of mechanical coupling.

Each of the pair of handcuffs 12 includes a lock housing 22 which has top side 24 and a bottom side 26; the top side 24 is concavely arcuate with respect to the bottom side 26 to conform to curvature of a respective detainee's wrist 14. The lock housing 22 has a lock slot 28 extending downwardly through the top side 24 and the lock slot 28 is elongated to extend between a first lateral side 30 and a second lateral side 32 of the lock housing 22. Each of the pair of handcuffs 12 includes a first cuff 34 which comprises a pair of members 36 each curving upwardly from the top side 24 from an intersection between the top side 24 and the second lateral side 32. The first cuff 34 curves toward the first lateral side 30 and each of the pair of members 36 is spaced apart from each other such that a space 38 between the pair of members 36 is aligned with the lock slot 28 in the top side 24 of the lock housing 22.

Each of the pair of handcuffs 12 includes a second cuff 40 which has a coupled end 42 that is pivotally coupled between a distal end 44 of each of the pair of members 36 of the first cuff 34. The second cuff 40 is curved between the coupled end 42 and a free end 46 of the second cuff 40. Additionally, the second cuff 40 has a plurality of teeth 48 each is integrated into a bottom edge 50 of the second cuff 40 and the teeth 48 are evenly spaced apart from each other and are distributed from the free end 46 of the second cuff 40 toward the coupled end 42 of the second cuff 40. The second cuff 40 is positionable in a closed position having the second cuff 40 extending into the lock slot 28 such that the first cuff 34 and the second cuff 40 forms a closed loop to enable the first cuff 34 and the second cuff 40 to extend around a respective detainee's wrist 14. Conversely, the second cuff 40 is positionable in an open position having the second cuff 40 being displaced from the lock slot 28 such that the first cuff 34 and the second cuff 40 defines an open loop to enable the first cuff 34 and the second cuff 40 to be removed from the respective detainee's wrist 14.

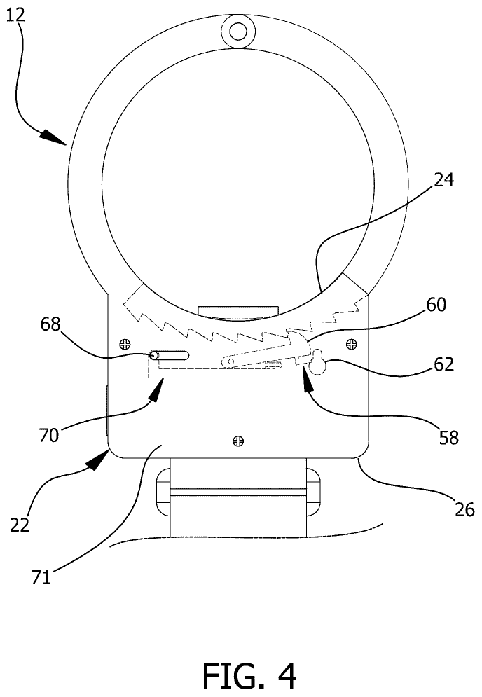

The chain 20 comprises a series of interlocking links 52 which are distributed between the bottom side 26 of the lock housing 22 of each of the pair of handcuffs 12 thereby attaching the lock housing 22 of each of the pair of handcuffs 12 together. Each of the series of interlocking links 52 may include a sleeve 54 and a resilient member 56 which forms a closed loop and which extends through the sleeve 54 associated with each of a pair of adjacent interlocking links 52. Each of the pair of handcuffs 12 includes a locking mechanism 58 which is movably integrated into the lock housing 22 thereby enabling the locking mechanism 58 to releasably engage respective teeth 48 on the bottom edge 50 of the second cuff 40 for releasably retaining the second cuff 40 in the closed position. As is most clearly shown in FIG. 4, the locking mechanism 58 may include a finger 60 pivotally disposed in the lock housing 22 which is biased to engage the respective teeth 48 and a key hole 62 that extends through a front side 64 of said lock housing 22 which insertably receives a key 66 that displaces the finger 60 from the respective teeth 48. The locking mechanism 58 may further include a double lock knob 68 that is slidably positioned in a channel 70 that extends through a back side 71 of the lock housing 22 which can be positioned in a locking position having the double lock knob 68 being positioned beneath the finger 60 to inhibit the finger 60 from disengaging the respective teeth 48.

A pair of heartbeat monitors 72 is provided and each of the pair of heartbeat monitors 72 is integrated into a respective one of the pair of handcuffs 12. In this way each of the pair of heartbeat monitors 72 can monitor the detainee's 16 heartbeat when the pair of handcuffs 12 are worn on the detainee's wrists 14. Each of the pair of heartbeat monitors 72 includes an audible alert which emits an audible alarm when a respective heartbeat monitor 72 detects a heartbeat that is displaying abnormalities. In this way the audible alert can audibly alert the law enforcement officer 18 that the detainee 16 is in need of medical attention.

Each of pair of heartbeat monitors 72 comprises a processing circuit 74 which is integrated into the lock housing 22 and the processing circuit 74 receives an alert input. Additionally, the processing circuit 74 includes an electronic memory 76 which stores digital information comprising a heartbeat analysis program 77. Each of the pair of heartbeat monitors 72 includes a pair of heartbeat sensors 78 which is each attached to the top side 24 of the lock housing 22. In this way each of the pair of heartbeat sensors 78 is in contact with the respective detainee's wrist 14 when the pair of handcuffs 12 are worn on the detainee's wrists 14 thereby enabling the pair of heartbeat sensors 78 to detect the detainee's 16 heartbeat.

Each of the pair of heartbeat sensors 78 is positioned on opposing sides of the lock slot 28 with respect to each other. Additionally, each of the pair of heartbeat sensors 78 is electrically coupled to the processing circuit 74. The processing circuit 74 receives the alert input when the heartbeat analysis program 77 determines that the pair of heartbeat sensors 78 senses a heartbeat that is displaying abnormalities. Each of the pair of heartbeat sensors 78 may comprise an optical sensor that employs infra-red light to sense the expansion and contraction of capillaries in the detainee's wrist 14 which results from the detainee's 16 heartbeat.

Each of the pair of heartbeat monitors 72 includes a light 80 which is attached to the front side 64 of the lock housing 22 to emit light outwardly from the lock housing 22 and the light 80 is electrically coupled to the processing circuit 74. The light 80 is turned on when the processing circuit 74 receives the alert input to visually alert the law enforcement officer 18 that the detainee 16 is in need of medical attention. The light 80 may comprise a light emitting diode or other type of electronic light emitter and the light 80 may emit light of a red color.

Each of the pair of heartbeat monitors 72 includes a speaker 82 which is integrated into the second lateral side 32 of the lock housing 22 and the speaker 82 emits an audible alert outwardly from the lock housing 22. The speaker 82 is electrically coupled to the processing circuit 74 and the speaker 82 comprises the audible alert of a respective heartbeat monitor 72. Furthermore, the speaker 82 is turned on when the processing circuit 74 receives the alert input to audibly alert the law enforcement officer 18 that the detainee 16 is in need of medical attention. The speaker 82 may comprise an electronic speaker of any conventional design that has an output intensity of at least 80.0 decibels. Each of the pair of heartbeat monitors 72 includes a vibration unit 83 that is integrated into the lock housing 22 and the vibration unit 83 vibrates the lock housing 22 when the vibration unit 83 is actuated. The vibration unit 83 is electrically coupled to the processing circuit 74 and the vibration unit 83 is actuated when the processing circuit 74 receives the alert input. Furthermore, the vibration unit 83 may comprise an electro-mechanical vibration unit of any conventional design that is capable of producing vibration that can be sensed by the detainee 16.

Each of the pair of heartbeat monitors 72 includes a power supply 84 which is integrated into the lock housing 22 and the power supply 84 is electrically coupled to the processing circuit 74. The power supply 84 comprises a rechargeable battery 86 which is positioned within the lock housing 22 and the rechargeable battery 86 is electrically coupled to the processing circuit 74. The power supply 84 includes a charge port 88 that is recessed into the front side 64 of the lock housing 22 and the charge port 88 is electrically coupled to the rechargeable battery 86. The charge port 88 insertably receives a charge cord 90 for charging the rechargeable battery 86. Additionally, the charge port 88 may comprise a micro universal serial bus port or other type of charge port commonly associated with rechargeable electronic devices.

In use, the pair of handcuffs 12 are placed on the detainee's wrists 14 to enable the law enforcement officer 18 to restrain the detainee 16. Each of the pair of heartbeat monitors 72 senses and monitors the detainee's 16 heartbeat while the detainee 16 is being restrained. The light 80 is actuated to emit light 80 and the speaker 82 is actuated to emit the audible alarm and the vibration unit 83 is actuated to vibrate the handcuffs 12 when the heartbeat monitors 72 determine that the detainee's 16 heartbeat is displaying abnormalities. In this way the law enforcement officer 18 is notified that the detainee 16 is in need of medical attention as soon as possible. Thus, the health and safety of the detainee 16 is increased while the detainee 16 is being restrained and transported to a law enforcement facility.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A heartbeat detecting handcuff device for detecting the heartbeat of a detainee and generating an alert when the detainee's heartbeat displays abnormalities, said device comprising:
a pair of handcuffs being configured to be placed on a detainee's wrists by a law enforcement officer for restraining the detainee, said pair of handcuffs including a chain which extends between said pair of handcuffs wherein said chain is configured to secure the detainee's wrists together when said pair of handcuffs is placed on the detainee's wrists; and
a pair of heartbeat monitors, each of said pair of heartbeat monitors being integrated into a respective one of said pair of handcuffs wherein said each of said pair of heartbeat monitors is configured to monitor the detainee's heartbeat when said pair of handcuffs are worn on the detainee's wrists, each of said pair of heartbeat monitors including an audible alert which emits an audible alarm when a respective heartbeat monitor detects a heartbeat that is displaying abnormalities wherein said audible alert is configured to audibly alert the law enforcement officer that the detainee is in need of medical attention.

2. The heartbeat detecting handcuff device according to claim 1, wherein:
each of said pair of handcuffs includes a lock housing which has top side and a bottom side;
said top side is concavely arcuate with respect to said bottom side wherein said top side is configured to conform to curvature of a respective detainee's wrist;
said lock housing has a lock slot extending downwardly through said top side;
said lock slot is elongated to extend between a first lateral side and a second lateral side of said lock housing;
each of said pair of handcuffs includes a first cuff which comprises a pair of members each curving upwardly from said top side from an intersection between said top side and said second lateral side;
said first cuff curves toward said first lateral side;
each of said pair of members is spaced apart from each other such that a space between said pair of members is aligned with said lock slot in said top side of said lock housing;
each of said pair of handcuffs includes a second cuff having a coupled end being pivotally coupled between a distal end of each of said pair of members of said first cuff;
said second cuff is curved between said coupled end and a free end of said second cuff;
said second cuff has a plurality of teeth each being integrated into a bottom edge of said second cuff;
said teeth are evenly spaced apart from each other and are distributed from said free end of said second cuff toward said coupled end of said second cuff; and
said chain comprises a series of interlocking links being distributed between said bottom side of said lock housing of each of said pair of handcuffs thereby attaching said lock housing of each of said pair of handcuffs together.

3. The heartbeat detecting handcuff device according to claim 2, wherein:
said second cuff is positionable in a closed position having said second cuff extending into said lock slot such that said first cuff and said second cuff forms a closed loop wherein said closed loop is configured to enable said first cuff and said second cuff to extend around a respective detainee's wrist; and
said second cuff is positionable in an open position having said second cuff being displaced from said lock slot such that said first cuff and said second cuff defines an open loop wherein said open loop is configured to enable said first cuff and said second cuff to be removed from the respective detainee's wrist.

4. The heartbeat detecting handcuff device according to claim 2, wherein each of said pair of handcuffs includes a locking mechanism being movably integrated into said lock housing thereby enabling said locking mechanism to releasably engage respective teeth on said bottom edge of said second cuff for releasably retaining said second cuff in said closed position.

5. The heartbeat detecting handcuff device according to claim 2, wherein:
each of pair of heartbeat monitors includes a processing circuit being integrated into said lock housing;
said processing circuit receives an alert input;
said processing circuit includes an electronic memory which stores digital information comprising a heartbeat analysis program; and
each of pair of heartbeat monitors includes a pair of heartbeat sensors;
each of said pair of heartbeat sensors is attached to said top side of said lock housing wherein each of said pair of heartbeat sensors is configured to be in contact with the respective detainee's wrist when said pair of handcuffs are worn on the detainee's wrists thereby enabling said pair of heartbeat sensors to detect the detainee's heartbeat;
each of said pair of heartbeat sensors is positioned on opposing sides of said lock slot with respect to each other;
each of said pair of heartbeat sensors is electrically coupled to said processing circuit; and
said processing circuit receives said alert input when said heartbeat analysis program determines that said pair of heartbeat sensors senses a heartbeat that is displaying abnormalities.

6. The heartbeat detecting handcuff device according to claim 5, wherein:
each of pair of heartbeat monitors includes a light being attached to a front side of said lock housing wherein said light is configured to emit light outwardly from said lock housing;
said light is electrically coupled to said processing circuit; and
said light is turned on when said processing circuit receives said alert input wherein said light is configured to visually alert the law enforcement officer that the detainee is in need of medical attention.

7. The heartbeat detecting handcuff device according to claim 5, wherein:
each of pair of heartbeat monitors includes a speaker being integrated into said second lateral side of said lock housing wherein said speaker is configured to emit an audible alert outwardly from said lock housing;
said speaker is electrically coupled to said processing circuit;
said speaker defining said audible alert of a respective heartbeat monitor; and
said speaker is turned on when said processing circuit receives said alert input wherein said speaker is configured to audibly alert the law enforcement officer that the detainee is in need of medical attention.

8. The heartbeat detecting handcuff device according to claim 5, wherein:
each of pair of heartbeat monitors includes a power supply being integrated into said lock housing;
said power supply is electrically coupled to said processing circuit; and
said power supply comprises:
a rechargeable battery being positioned within said lock housing, said rechargeable battery being electrically coupled to said processing circuit; and
a charge port being recessed into said front side of said lock housing, said charge port being electrically coupled to said rechargeable battery, said charge port insertably receiving a charge cord for charging said rechargeable battery.

9. The device according to claim 5, wherein:
each of said pair of heartbeat monitors includes a vibration unit being integrated into said lock housing;
said vibration unit vibrating said lock housing when said vibration unit is actuated;
said vibration unit being electrically coupled to said processing circuit;
said vibration unit being actuated when said processing circuit receives said alert input.

9. A heartbeat detecting handcuff device for detecting the heartbeat of a detainee and generating an alert when the detainee's heartbeat displays abnormalities, said device comprising:
a pair of handcuffs being configured to be placed on a detainee's wrists by a law enforcement officer for restraining the detainee, said pair of handcuffs including a chain which extends between said pair of handcuffs wherein said chain is configured to secure the detainee's wrists together when said pair of handcuffs is placed on the detainee's wrists, each of said pair of handcuffs including a lock housing which has top side and a bottom side, said top side being concavely arcuate with respect to said bottom side wherein said top side is configured to conform to curvature of a respective detainee's wrist, said lock housing having a lock slot extending downwardly through said top side, said lock slot being elongated to extend between a first lateral side and a second lateral side of said lock housing, each of said pair of handcuffs including a first cuff which comprises a pair of members each curving upwardly from said top side from an intersection between said top side and said second lateral side, said first cuff curving toward said first lateral side, each of said pair of members being spaced apart from each other such that a space between said pair of members is aligned with said lock slot in said top side of said lock housing, each of said pair of handcuffs including a second cuff having a coupled end being pivotally coupled between a distal end of each of said pair of members of said first cuff, said second cuff being curved between said coupled end and a free end of said second cuff, said second cuff having a plurality of teeth each being integrated into a bottom edge of said second cuff, said teeth being evenly spaced apart from each other and being distributed from said free end of said second cuff toward said coupled end of said second cuff, said second cuff being positionable in a closed position having said second cuff extending into said lock slot such that said first cuff and said second cuff forms a closed loop wherein said closed loop is configured to enable said first cuff and said second cuff to extend around a respective detainee's wrist, said second cuff being positionable in an open position having said second cuff being displaced from said lock slot such that said first cuff and said second cuff defines an open loop wherein said open loop is configured to enable said first cuff and said second cuff to be removed from the respective detainee's wrist, said chain comprising a series of interlocking links being distributed between said bottom side of said lock housing of each of said pair of handcuffs thereby attaching said lock housing of each of said pair of handcuffs together, each of said pair of handcuffs including a locking mechanism being movably integrated into said lock housing thereby enabling said locking mechanism to releasably engage respective teeth on said bottom edge of said second cuff for releasably retaining said second cuff in said closed position; and
a pair of heartbeat monitors, each of said pair of heartbeat monitors being integrated into a respective one of said pair of handcuffs wherein said each of said pair of heartbeat monitors is configured to monitor the detainee's heartbeat when said pair of handcuffs are worn on the detainee's wrists, each of said pair of heartbeat monitors including an audible alert which emits an audible alarm when a respective heartbeat monitor detects a heartbeat that is displaying abnormalities wherein said audible alert is configured to audibly alert the law enforcement officer that the detainee is in need of medical attention, each of pair of heartbeat monitors comprising:
a processing circuit being integrated into said lock housing, said processing circuit receiving an alert input, said processing circuit including an electronic memory which stores digital information comprising a heartbeat analysis program;
a pair of heartbeat sensors, each of said pair of heartbeat sensors being attached to said top side of said lock housing wherein each of said pair of heartbeat sensors is configured to be in contact with the respective detainee's wrist when said pair of handcuffs are worn on the detainee's wrists thereby enabling said pair of heartbeat sensors to detect the detainee's heartbeat, each of said pair of heartbeat sensors being positioned on opposing sides of said lock slot with respect to each other, each of said pair of heartbeat sensors being electrically coupled to said processing circuit, said processing circuit receiving said alert input when said heartbeat analysis program determines that said pair of heartbeat sensors senses a heartbeat that is displaying abnormalities;
a light being attached to a front side of said lock housing wherein said light is configured to emit light outwardly from said lock housing, said light being electrically coupled to said processing circuit, said light being turned on when said processing circuit receives said alert input wherein said light is configured to visually alert the law enforcement officer that the detainee is in need of medical attention;
a speaker being integrated into said second lateral side of said lock housing wherein said speaker is configured to emit an audible alert outwardly from said lock housing, said speaker being electrically coupled to said processing circuit, said speaker defining said audible alert of a respective heartbeat monitor, said speaker being turned on when said processing circuit receives said alert input wherein said speaker is configured to audibly alert the law enforcement officer that the detainee is in need of medical attention;
a vibration unit being integrated into said lock housing, said vibration unit vibrating said lock housing when said vibration unit is actuated, said vibration unit being electrically coupled to said processing circuit, said vibration unit being actuated when said processing circuit receives said alert input; and
a power supply being integrated into said lock housing, said power supply being electrically coupled to said processing circuit, said power supply comprising:
a rechargeable battery being positioned within said lock housing, said rechargeable battery being electrically coupled to said processing circuit; and
a charge port being recessed into said front side of said lock housing, said charge port being electrically coupled to said rechargeable battery, said charge port insertably receiving a charge cord for charging said rechargeable battery.
